(19) United States
(12) Patent Application Publication
(10) Pub. No.: US 2026/0240658 A1
(43) Pub. Date: Aug. 20, 2026

(54) THREADED IMPLANTS AND METHODS OF USE ACROSS BONE SEGMENTS

(71) Applicant: SI-Bone Inc., Santa Clara, CA (US)

(72) Inventors: Derek P. LINDSEY, San Jose, CA (US); Paul M. SAND, Redwood City, CA (US); Bret W. SCHNEIDER, San Jose, CA (US); Scott A. YERBY, Montara, CA (US); Nikolas F. KERR, Milpitas, CA (US); Regina MACBARB, Carlsbad, CA (US)

(21) Appl. No.: 19/317,804

(22) Filed: Sep. 3, 2025

Related U.S. Application Data
(63) Continuation of application No. 18/066,872, filed on Dec. 15, 2022, now Pat. No. 12,427,028.
Application No. 18/066,872 is a continuation of application No. 16/368,686, filed on Mar. 28, 2019, now abandoned.
(60) Provisional application No. 62/649,466, filed on Mar. 28, 2018.

(51) Int. Cl. A61F 2/30; A61B 17/80; A61F 2/28; A61F 2/46

(52) U.S. Cl. CPC A61F 2/30988; A61B 17/80; A61F 2/28; A61F 2/30; A61F 2/4603; A61F 2002/2835; A61F 2002/30011; A61F 2002/30156; A61F 2002/30289; A61F 2002/30405; A61F 2002/30622; A61F 2002/30784; A61F 2002/30858; A61F 2002/30868; A61F 2002/30995

(57) ABSTRACT

A threaded implant can be provided with an elongated main body having external threads configured to thread into bone, and an internal support structure located within the external threads. The internal support structure has a helical arrangement that extends in an opposite direction to the external threads. Other threaded implants and methods are also disclosed.

### CROSS REFERENCE TO RELATED APPLICATIONS

This application is a continuation of U.S. application Ser. No. 18/066,872, filed Dec. 15, 2022, which is a continuation of U.S. application Ser. No. 16/368,686, filed Mar. 28, 2019, now abandoned, which claims the benefit of U.S. Provisional Application No. 62/649,466 filed Mar. 28, 2018, each of which are herein incorporated by reference in its entirety.

### INCORPORATION BY REFERENCE

All publications and patent applications mentioned in this specification are herein incorporated by reference for all intents and purposes to the same extent as if each individual publication or patent application was specifically and individually indicated to be incorporated by reference.

### FIELD

Embodiments of the disclosure relate generally to fixation or fusion of a bone joint or fracture, and more specifically, to threaded devices and methods of implanting the devices across bone segments.

### BACKGROUND

Sacroiliac joint (SI-Joint) fusion is a surgical procedure that is performed to alleviate pain coming from the SI-Joint in patients who have failed to receive adequate pain relief with non-surgical treatments of the SI-Joint. Some conditions of the SI-Joint that may be treated with SI-Joint fusion (arthrodesis) are: degenerative sacroiliitis, inflammatory sacroiliitis, iatrogenic instability of the sacroiliac joint, osteitis condensans ilii, or traumatic fracture dislocation of the pelvis. Historically, screws and screws with plates were used as the standard instrumentation for sacro-iliac fusion. An SI-Joint fusion consisted of an open surgical approach to the SI-Joint from an anterior, a posterior, or a lateral direction. The surgeon would then debride (remove) the cartilage from the articular portion of the joint and the interosseous ligament from the fibrous portion of the joint. These open approaches require a large incision and deep soft tissue dissection to approach the damaged, subluxed, dislocated, fractured, or degenerative SI-Joint.

With more recent advancements in SI-Joint surgery, a typical technique for placing implants involves placement of one or multiple implants from a lateral to medial direction across the SI-Joint. These implants are placed with a starting point on the lateral aspect of the ilium. The implants are then directed across the ilium, across the sacroiliac joint and into the sacrum.

Various styles of implants are available today for fusing the SI-Joint and other joints in the above minimally invasive surgeries. However, it would be desirable to provide improved implants and methods to promote even faster and stronger fusion of bone joints.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIGS. 1 and 2 are, respectively, anterior and posterior anatomic views of the human hip girdle comprising the sacrum and the hip bones (the right ilium, and the left ilium), the sacrum being connected with both hip bones at the sacroiliac joint (in shorthand, the SI-Joint).

FIGS. 3 and 4 are embodiments of various implants that can be used for the fusion or fixation of a joint or two bone segments.

FIG. 5 illustrates an axial section view of the SI-Joint with an implant for the fixation of the SI-Joint using a lateral approach that goes laterally through the ilium, the SI-Joint, and into the sacrum S1.

FIG. 6 illustrates an axial section view of the SI-Joint with an implant for the fixation of the SI-Joint using a postero-lateral approach entering from the posterior iliac spine of the ilium, angling through the SI-Joint, and terminating in the sacral alae.

FIGS. 7A-7D are side section views of the formation of a broached bore in bone.

FIGS. 7E and 7F illustrate the assembly of a soft tissue protector system for placement over a guide wire.

FIGS. 8 to 10 are anatomic views showing, respectively, a pre-implanted perspective, implanted perspective, and implanted anterior view, the implantation of three implant structures for the fixation of the SI-Joint using a lateral approach through the ilium, the SI-Joint, and into the sacrum.

FIG. 11 is a perspective view showing an exemplary embodiment of a threaded implant constructed according to aspects of the present disclosure.

FIG. 12 is a side elevation exploded view of the implant shown in FIG. 11.

FIG. 13 is a side elevation cross-sectional view of the implant shown in FIG. 11.

FIG. 14 is a perspective view showing another exemplary embodiment of a threaded implant constructed according to aspects of the present disclosure.

FIG. 15 is a perspective exploded view of the implant shown in FIG. 14.

FIG. 16 is a side elevation cross-sectional view of the implant shown in FIG. 14.

FIG. 17 is a distal end view of the implant shown in FIG. 14.

FIG. 18 is a perspective view showing another exemplary embodiment of a threaded implant constructed according to aspects of the present disclosure.

FIG. 19 is a side elevation exploded view of the implant shown in FIG. 18.

FIG. 20 is a side elevation cross-sectional view of the implant shown in FIG. 18.

FIG. 21 is a distal end view of the implant shown in FIG. 18.

FIG. 22 is a perspective view showing another exemplary embodiment of a threaded implant constructed according to aspects of the present disclosure.

FIG. 23 is an opposite perspective view of the implant shown in FIG. 22.

FIG. 24 is a proximal end view of the implant shown in FIG. 22.

FIG. 25 is a distal end view of the implant shown in FIG. 22.

FIG. 26 is a perspective exploded view of the implant shown in FIG. 22.

FIG. 27 is a side elevation exploded view of the implant shown in FIG. 22.

### SUMMARY OF THE DISCLOSURE

According to aspects of the present disclosure, a threaded implant can be provided with an elongated main body having external threads configured to thread into bone, and an internal support structure located within the external threads. The internal support structure has a helical arrangement that extends in an opposite direction to the external threads. The threaded implant may further include a drive socket located on a proximal end of the main body and configured to receive a tip of a drive tool for rotationally driving the implant into bone. In some embodiments, a through bore is provided along a central longitudinal axis of the main body from a proximal end to a distal end. The external threads may have a single start and the internal support structure may have four starts. In some embodiments, the internal support structure has a pitch that is eight time the pitch of the external threads. The implant may further include fenestrations or interstices between the external threads and the internal support structure. The fenestrations or interstices may be filled with a porous infill which provides scaffolding with increased surface area for new bone growth. In some embodiments, the porous infill is at least 20% more porous than the external threads and the internal support structure.

According to other aspects of the present disclosure, a threaded implant can be provided with an elongated main body, a first set of threads, a second set of threads and a sleeve. The main body has a proximal end, a mid-section and a distal end. The first set of threads is provided along the proximal end of the main body and the second set of threads is provided along the distal end. The sleeve is located on the mid-section of the main body such that it may rotate with respect to the main body while being constrained from axial movement. In some embodiments, the sleeve has a transverse cross-section that is rectilinear, has at least one apex and/or is triangular in shape. The second set of threads may have a pitch that is greater than a pitch of the first set of threads.

According to other aspects of the present disclosure, a threaded implant can be provided with an elongated main body, a head portion and a threaded portion. The elongated main body has a proximal end, a mid-section and a distal end. The head portion is located on the proximal end of the main body and configured to abut against an outer surface of a bone segment. The threaded portion is located on the distal end of the main body and has a transverse cross-section that is triangular in shape. In some embodiments, the implant is provided a drive socket located on the proximal end of the main body and configured to receive a tip of a drive tool for rotationally driving the implant into bone.

According to other aspects of the present disclosure, a threaded implant can be provided with an elongated main body, a proximal screw, a proximal screw cap and at least one rotation stop. The elongated main body has a proximal end, a mid-section and a distal end.

### DETAILED DESCRIPTION

A joint of a patient can be decorticated or selectively decorticated in order to promote bone regeneration and fusion at the implant site. Many types of hardware are available both for the fixation of bones that are fractured and for the fixation of bones that are to be fused (arthrodesed). While the following examples focus on the SI-Joint, the methods, instrumentation and implants disclosed herein may be used for decortication of other body joints as well.

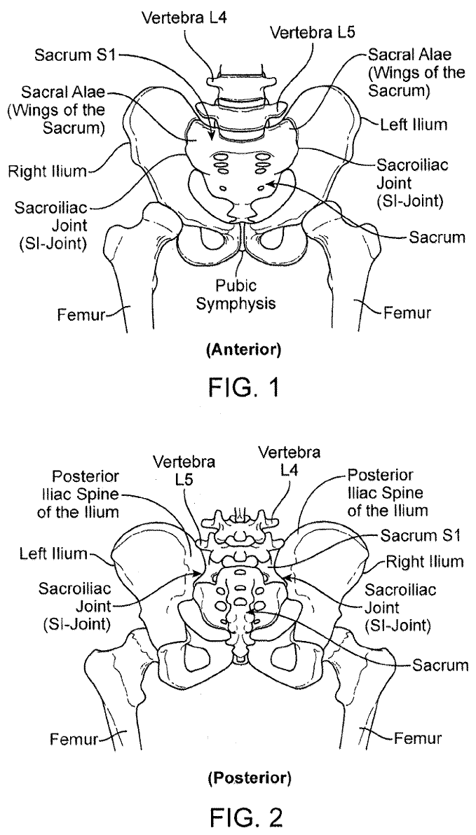

Referring to FIGS. 1 and 2, the human hip girdle is made up of three large bones joined by three relatively immobile joints. One of the bones is called the sacrum and it lies at the bottom of the lumbar spine, where it connects with the L5 vertebra. The other two bones are commonly called “hip bones” and are technically referred to as the right ilium and-the left ilium. The sacrum connects with both hip bones at the sacroiliac joint (in shorthand, the SI-Joint).

The SI-Joint functions in the transmission of forces from the spine to the lower extremities, and vice-versa. The SI-Joint has been described as a pain generator for up to 22% of lower back pain patients.

To relieve pain generated from the SI-Joint, sacroiliac joint fusion is typically indicated as surgical treatment, e.g., for degenerative sacroiliitis, inflammatory sacroiliitis, iatrogenic instability of the sacroiliac joint, osteitis condensans ilii, or traumatic fracture dislocation of the pelvis. In some currently performed procedures, screws or screws with plates are used for sacro-iliac fusion. At the time of the procedure, articular cartilage may be removed from the “synovial joint” portion of the SI-Joint. This can require a large incision to approach the damaged, subluxed, dislocated, fractured, or degenerated joint. The large incision and removal of tissue can cause significant trauma to the patient, resulting in pain and increasing the time to heal after surgery.

In addition, screw type implants tend to be susceptible to rotation and loosening, especially in joints that are subjected to torsional forces, such as the SI-Joint. Excessive movement of the implant after implantation may result in the failure of the implant to incorporate and fuse with the bone, which may result in the need to remove and replace the failed implant.

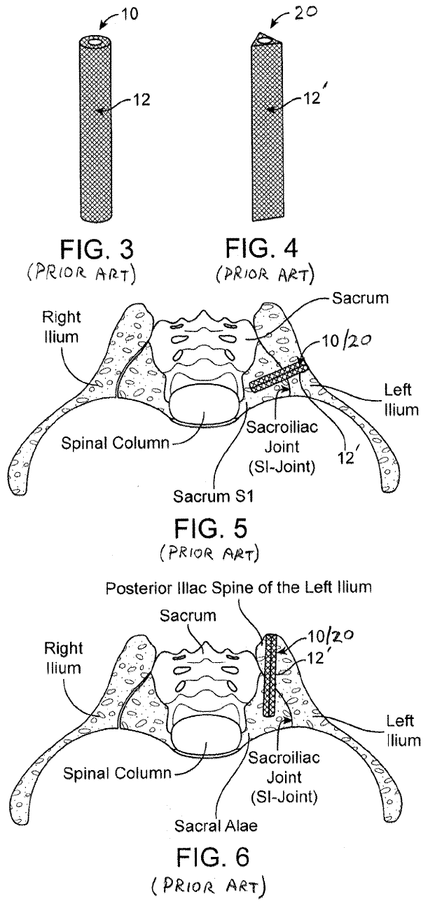

FIG. 3 and FIG. 4 illustrate straight implants 10 and 20, respectively, with a solid elongate body 12 or 12′ that can be used for the fixation or fusion of two bone segments. The implant 10 shown in FIG. 3 is cylindrical and can optionally have screw threads along the exterior of the implant body. As mentioned above, cylindrical screw type implants can suffer from excessive rotation. One solution to this problem is the implant 20 in FIG. 4, which has a non-cylindrical cross-sectional area. For example, as shown, the implant 20 can have a triangular cross-sectional area, although other rectilinear cross-sectional profiles may be used as well, including rectangular, hexagonal and the like. Non-cylindrical implants need not have a strict rectilinear cross-sectional profile in order to resist rotation. A cross-sectional area that is non-circular will generally suffice. For example, a tear drop shaped cross-sectional area, or a cross-sectional area with at least one apex, can resist rotation. Other non-circular cross-sectional geometries that may not have a rectilinear component can also work, such as oval cross-sections.

FIG. 5 illustrates insertion of the implant 10 or 20 of FIG. 3 or FIG. 4 across the SI-Joint using a lateral approach that goes laterally through the ilium, across the SI-Joint, and into the sacrum. FIG. 6 illustrates insertion of the same implant across the SI-Joint using a postero-lateral approach entering from the posterior iliac spine of the ilium, angling through the SI-Joint, and terminating in the sacral alae. The implants and instrumentation described herein typically can be inserted across the SI-Joint according to one of these two approaches, or with a similar approach.

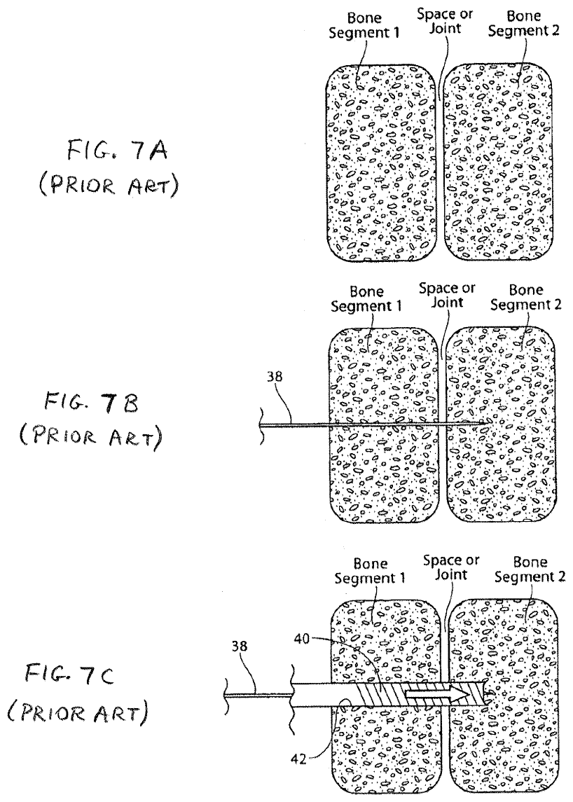

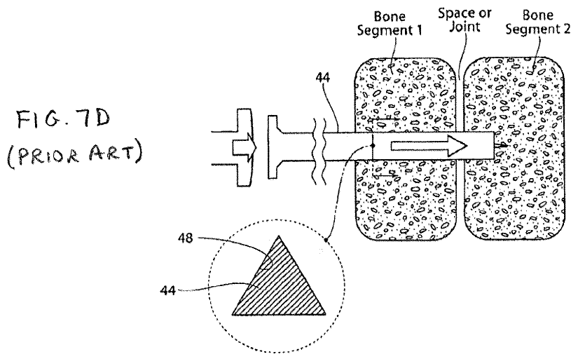

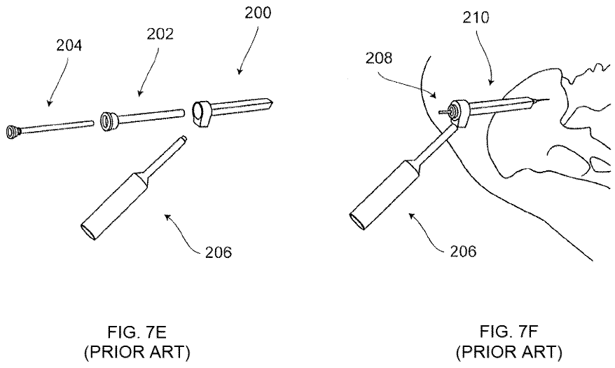

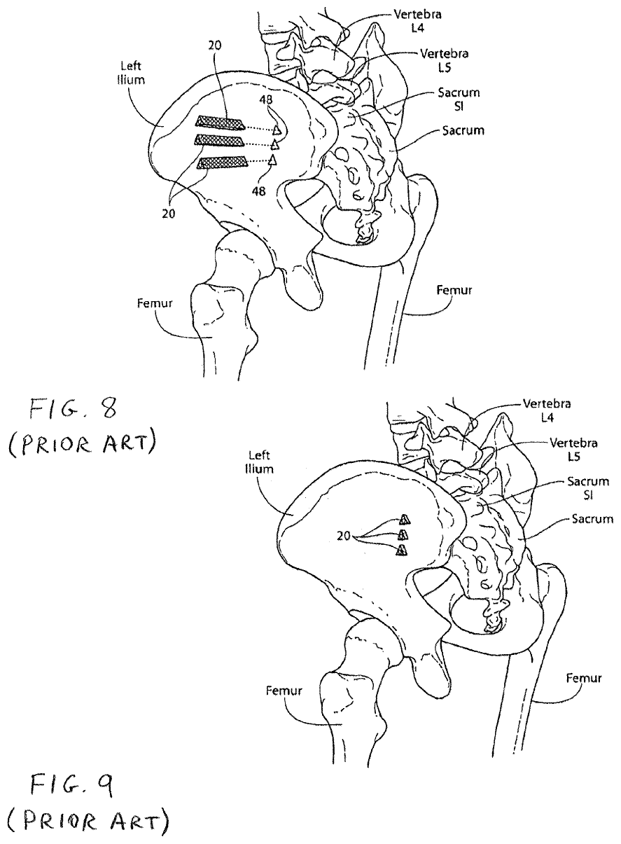

Referring to FIGS. 7-10, an exemplary method for fixation of the SI-Joint will be described. Elongated, stem-like implant structures 10 or 20 like those shown in FIGS. 3 and 4 make possible the fixation of the SI-Joint in a minimally invasive manner. These implant structures can be effectively implanted through the use a lateral surgical approach (as shown in FIG. 5). The procedure may be aided by conventional lateral, inlet, and outlet visualization techniques, e.g., using X-ray image intensifiers such as a C-arms or fluoroscopes to produce a live image feed, which is displayed on a TV screen.

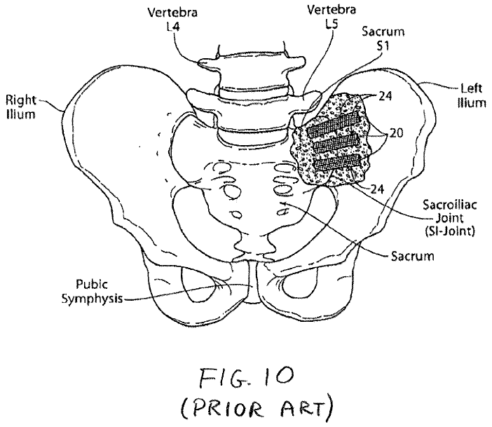

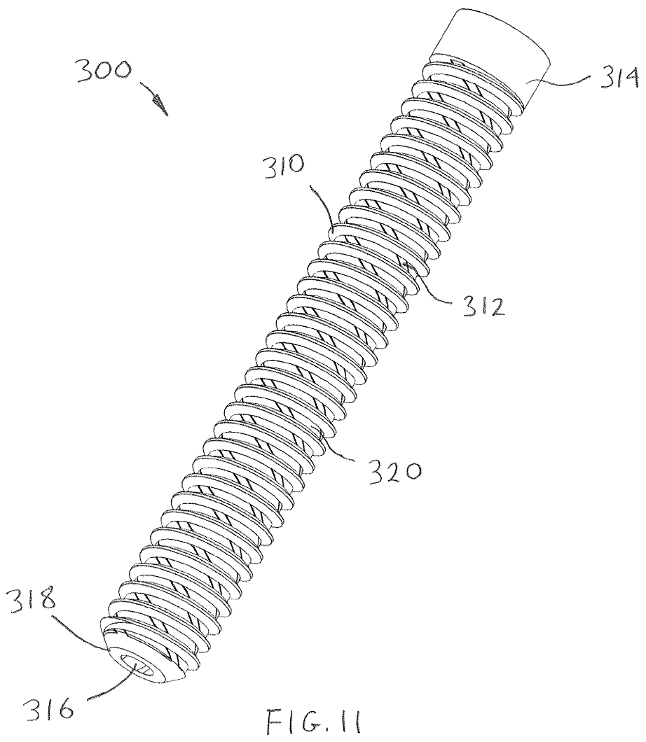

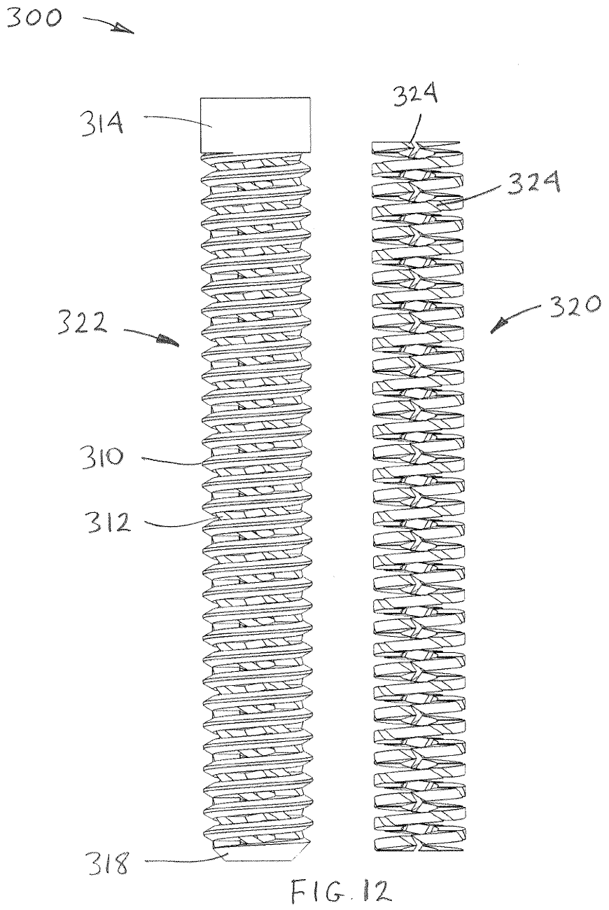

In this exemplary embodiment, one or more implant structures 20 are introduced laterally through the ilium, the SI-Joint, and into the sacrum. This path and resulting placement of the implant structure(s) 20 are best shown in FIGS. 9 and 10. In the illustrated embodiment, three implant structures 20 are placed in this manner. Also in the illustrated embodiment, the implant structures 20 are rectilinear in cross section and triangular in this case, but it should be appreciated that implant structures 20 of other rectilinear cross sections can be used.

Additionally, in some procedures (not discussed in further detail herein), implants may be introduced into the SI-Joint from an anterior direction. Further information on anterior techniques may be found in co-pending U.S. patent application Pub. No. 2015/0105828 filed Oct. 15, 2014 and entitled “Implant Placement”. The decortication instruments and methods disclosed herein and variants thereof may also be utilized in these anterior procedures.

Before undertaking a lateral implantation procedure, the physician diagnoses the SI-Joint segments that are to be fixated or fused (arthrodesed) using, e.g., the Fortin finger test, thigh thrust, FABER, Gaenslen's, compression, distraction, and or diagnostic SI-Joint injection.

Aided by lateral, inlet, and outlet C-arm views, and with the patient lying in a prone position, the physician aligns the greater sciatic notches and then the alae (using lateral visualization) to provide a true lateral position. A 3 cm incision is made starting aligned with the posterior cortex of the sacral canal, followed by blunt tissue separation to the ilium. From the lateral view, the guide pin 38 (with pin sleeve (not shown)) (e.g., a Steinmann Pin) is started resting on the ilium at a position inferior to the sacrum end plate and just anterior to the sacral canal. In the outlet view, the guide pin 38 should be parallel to the sacrum end plate at a shallow angle anterior (e.g., 15 degree to 20 degree off the floor, as FIG. 10 shows). In a lateral view, the guide pin 38 should be posterior to the sacrum anterior wall. In the outlet view, the guide pin 38 should be superior to the first sacral foramen and lateral of mid-line. This corresponds generally to the sequence shown diagrammatically in FIGS. 7A and 7B. A soft tissue protector (not shown), and a drill sleeve (not shown) within the soft tissue protector, may be slipped over the guide pin 38 and firmly against the ilium before removing the guide pin sleeve (not shown).

Over the guide pin 38 (and through the soft tissue protector and drill sleeve), a pilot bore 42 may be drilled with cannulated drill bit 40, as is diagrammatically shown in FIG. 7C. The pilot bore 42 may extend through the ilium, through the SI-Joint, and into the sacrum. The drill bit 40 and drill sleeve (not shown) are then removed.

A shaped broach 44 may be tapped into the pilot bore 42 over the guide pin 38 (and through the soft tissue protector, not shown) to create a broached bore 48 with the desired profile for the implant structure 20, which, in the illustrated embodiment, is triangular. This generally corresponds to the sequence shown diagrammatically in FIG. 7D. The triangular profile of the broached bore 48 is also shown in FIG. 8.

FIGS. 7E and 7F illustrate an embodiment of the assembly of a soft tissue protector or dilator or delivery sleeve 200 with a drill sleeve 202, a guide pin sleeve 204 and a handle 206. In some embodiments, the drill sleeve 202 and guide pin sleeve 204 can be inserted within the soft tissue protector 200 to form a soft tissue protector assembly 210 that can slide over the guide pin 208 until bony contact is achieved. The soft tissue protector 200 can be any one of the soft tissue protectors or dilators or delivery sleeves disclosed herein. In some embodiments, an expandable dilator or delivery sleeve 200 can be used in place of a conventional soft tissue dilator. In the case of the expandable dilator, in some embodiments, the expandable dilator can be slid over the guide pin and then expanded before the drill sleeve 202 and/or guide pin sleeve 204 are inserted within the expandable dilator. In other embodiments, insertion of the drill sleeve 202 and/or guide pin sleeve 204 within the expandable dilator can be used to expand the expandable dilator.

In some embodiments, a dilator can be used to open a channel though the tissue prior to sliding the soft tissue protector assembly 210 over the guide pin. The dilator(s) can be placed over the guide pin, using for example a plurality of sequentially larger dilators or using an expandable dilator. After the channel has been formed through the tissue, the dilator(s) can be removed and the soft tissue protector assembly can be slid over the guide pin. In some embodiments, the expandable dilator can serve as a soft tissue protector after being expanded. For example, after expansion the drill sleeve and guide pin sleeve can be inserted into the expandable dilator.

As shown in FIGS. 8 and 9, a triangular implant structure 20 can be now tapped through the soft tissue protector over the guide pin 38 through the ilium, across the SI-Joint, and into the sacrum, until the proximal end of the implant structure 20 is flush against the lateral wall of the ilium (see also FIGS. 5 and 10). The guide pin 38 and soft tissue protector are withdrawn, leaving the implant structure 20 residing in the broached passageway, flush with the lateral wall of the ilium (see FIGS. 5 and 10). In the illustrated embodiment, two additional implant structures 20 are implanted in this manner, as FIG. 9 best shows. In other embodiments, the proximal ends of the implant structures 20 are left proud of the lateral wall of the ilium, such that they extend 1, 2, 3 or 4 mm outside of the ilium. This ensures that the implants 20 engage the hard cortical portion of the ilium rather than just the softer cancellous portion, through which they might migrate if there was no structural support from hard cortical bone. The hard cortical bone can also bear the loads or forces typically exerted on the bone by the implant 20.

The implant structures 20 are sized according to the local anatomy. For the SI-Joint, representative implant structures 20 can range in size, depending upon the local anatomy, from about 35 mm to about 60 mm in length, and about a 7 mm inscribed diameter (i.e. a triangle having a height of about 10.5 mm and a base of about 12 mm). The morphology of the local structures can be generally understood by medical professionals using textbooks of human skeletal anatomy along with their knowledge of the site and its disease or injury. The physician is also able to ascertain the dimensions of the implant structure 20 based upon prior analysis of the morphology of the targeted bone using, for example, plain film x-ray, fluoroscopic x-ray, or MRI or CT scanning.

Using a lateral approach, one or more implant structures 20 can be individually inserted in a minimally invasive fashion across the SI-Joint, as has been described. Conventional tissue access tools, obturators, cannulas, and/or drills can be used for this purpose. Alternatively, the novel tissue access tools described above and in U.S. Provisional Patent Application No. 61/609,043, titled “TISSUE DILATOR AND PROTECTOR” and filed Mar. 9, 2012, and in U.S. Published Application No. 2017/0007409, titled “SYSTEMS, DEVICES, AND METHODS FOR JOINT FUSION” and filed Jul. 12, 2016, can also be used. No joint preparation, removal of cartilage, or scraping are required before formation of the insertion path or insertion of the implant structures 20, so a minimally invasive insertion path sized approximately at or about the maximum outer diameter of the implant structures 20 can be formed.

The implant structures 20 can obviate the need for autologous bone graft material, additional pedicle screws and/or rods, hollow modular anchorage screws, cannulated compression screws, threaded cages within the joint, or fracture fixation screws. Still, in the physician's discretion, bone graft material and other fixation instrumentation can be used in combination with the implant structures 20.

In a representative procedure, one to six, or perhaps up to eight, implant structures 20 can be used, depending on the size of the patient and the size of the implant structures 20. After installation, the patient would be advised to prevent or reduce loading of the SI-Joint while fusion occurs. This could be about a six to twelve week period or more, depending on the health of the patient and his or her adherence to post-op protocol.

The implant structures 20 make possible surgical techniques that are less invasive than traditional open surgery with no extensive soft tissue stripping. The lateral approach to the SI-Joint provides a straightforward surgical approach that complements the minimally invasive surgical techniques. The profile and design of the implant structures 20 minimize or reduce rotation and micromotion. Rigid implant structures 20 made from titanium provide immediate post-op SI-Joint stability. A bony in-growth region 24 comprising a porous plasma spray coating with irregular surface supports stable bone fixation/fusion. The implant structures 20 and surgical approaches make possible the placement of larger fusion surface areas designed to maximize post-surgical weight bearing capacity and provide a biomechanically rigorous implant designed specifically to stabilize the heavily loaded SI-Joint. In some embodiments, a fenestrated matrix implant may be used, providing cavities in which to pack bone growth material, and or providing additional surface area for bone on-growth, in-growth and or through-growth.

To improve the stability and weight bearing capacity of the implant, the implant can be inserted across three or more cortical walls. For example, after insertion the implant can traverse two cortical walls of the ilium and at least one cortical wall of the sacrum. The cortical bone is much denser and stronger than cancellous bone and can better withstand the large stresses found in the SI-Joint. By crossing three or more cortical walls, the implant can spread the load across more load bearing structures, thereby reducing the amount of load borne by each structure. In addition, movement of the implant within the bone after implantation is reduced by providing structural support in three locations around the implant versus two locations.

Further details of bone joint implants and methods of use can be found in U.S. Pat. No. 8,308,779 entitled “SYSTEMS AND METHODS FOR THE FIXATION OR FUSION OF BONE” filed Feb. 25, 2008, U.S. Pat. No. 7,922,765 entitled “SYSTEMS AND METHODS FOR THE FIXATION OR FUSION OF BONE” filed Mar. 24, 2005, U.S. Pat. No. 8,986,348 entitled “SYSTEMS AND METHODS FOR THE FUSION OF THE SACRAL-ILIAC JOINT” filed Oct. 5, 2010, and U.S. Pat. No. 8,414,648 entitled “APPARATUS, SYSTEMS, AND METHODS FOR ACHIEVING TRANS-ILIAC LUMBAR FUSION” filed Dec. 6, 2010 In the previously described methods, the implant(s) 10 or 20 (FIGS. 3 and 4) may be placed in the implant bore(s) 48 (FIG. 8) using a generally medial or axial, non-rotational force, such as tapping an implant into place using a slide hammer. In other embodiments, according to aspects of the present disclosure, the implant(s) may comprise external threads and may be threaded into place by applying a rotational force, as will now be described.

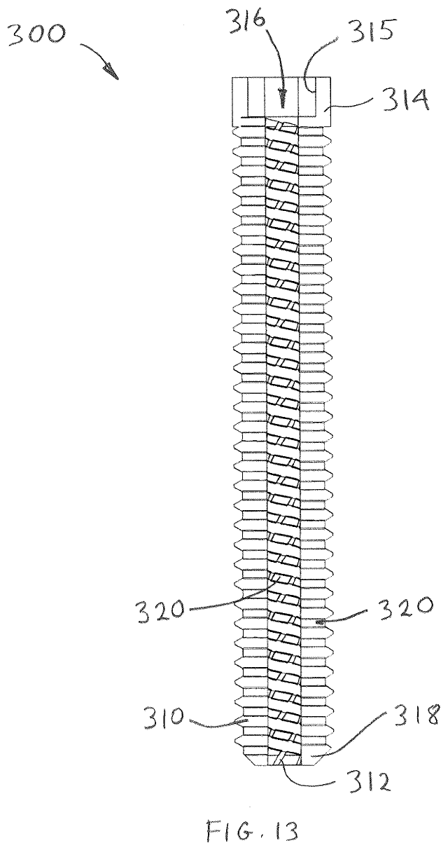

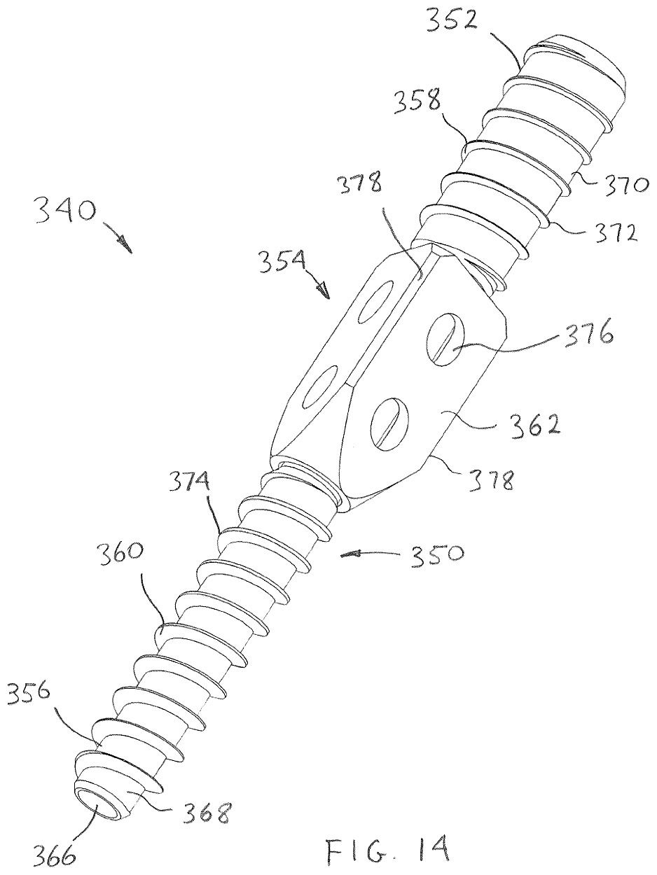

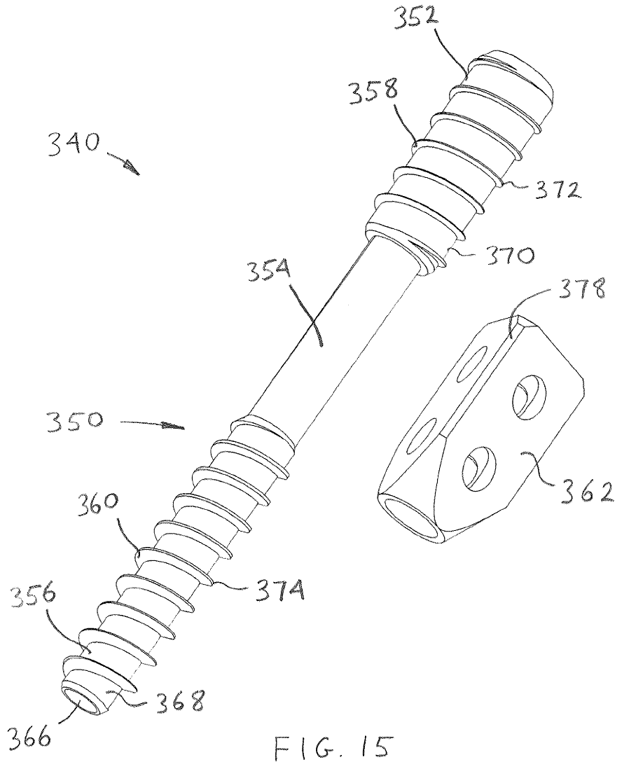

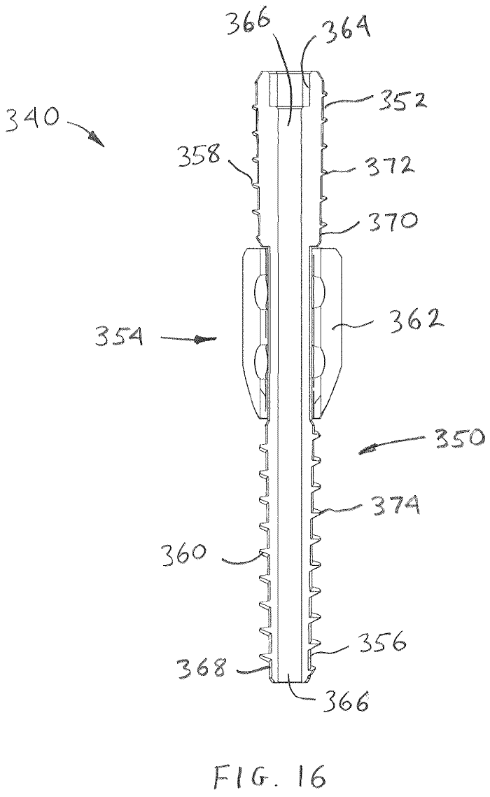

Referring to FIGS. 11-13, an exemplary embodiment of a threaded implant system constructed according to aspects of the present disclosure is shown. Implant 300 is provided with external threads 310 and an internal counter-rotating support structure 312. As best seen in FIG. 13, proximal end 314 may be provided with a hexagonally shaped socket 315 for receiving a drive tool (not shown) when implant 300 is being inserted or removed. A through bore 316 may be provided along the central axis from the proximal end 314 to the distal end 318.

External threads 310 are used to engage bone when threading implant 300 across a bone joint. In some embodiments, external threads 310 are self-tapping. In this embodiment, internal counter-rotating support structure 312 extends helically in an opposite direction from external threads 310 as shown. Support structure 312 provides stiffness and torsional rigidity to implant 300 while permitting fenestrations between external threads 310 for promoting better bony on-growth, ingrowth and/or through-growth. In this exemplary embodiment, a single external thread 310 helically extends from near the proximal end 314 to the distal end 318 of implant 300. In other embodiments (not shown), multiple starts of external threads 310 may be employed. In this embodiment, internal counter-rotating support structure 312 comprises four starts. In other embodiments (not shown), fewer or more starts may be employed. In this embodiment, internal counter-rotating support structure 312 has a pitch that is eight times the pitch of external threads 310. In other embodiments (not shown), the pitch of structure 312 may be less or more than eight times that of threads 310. In other embodiments (not shown), support structure 312 may extend in the same direction as external threads 310 rather than counter-rotating relative to it, and or may form a shape other than helical. Support structure 312 may comprise layers. In some embodiments, these layers alternate in direction.

In the exemplary embodiment of FIGS. 11-13, the fenestrations or interstices between external threads 310 and internal counter-rotating support structure 312 are filled with porous infill 320. Infill 320 provides scaffolding with a large surface area for new bone growth. Infill 320 can also add additional strength to implant 300 in compression, tension, torsion, bending, shear, etc., but still allow for better bony on-growth, ingrowth and/or through-growth than if the fenestrations or interstices were completely filled with less porous material. The exploded view of FIG. 12 shows main portion 322 of implant 300 separately from the porous infill 320 for clarity, although in this embodiment infill 320 is a collection of many individual segments rather than an interconnected structure. In some embodiments, main portion 322 may also be porous, but having a different porosity than infill 320. For example, infill 320 may be at least 20% more porous than main portion 322. Both may be made together in the same manufacturing process, such as 3D printing or other additive manufacturing process. In some embodiments, infill 320 is formed from the same material as main portion 322, while in other embodiments a different material or materials may be used to form infill 320.

In some embodiments, porous infill 320 has the same inner diameter as that of the main portion 322 of implant 300, forming a continuous surface defining through bore 316, as shown in FIG. 13. The outer diameter of infill 320 may be the same diameter of the radially inward end of the tapered portions of threads 310, thereby forming the root diameter of threads 310. The inner and outer diameters of counter-rotating support structure 312 may also be the same as those of infill 320, such that breaks 324 are formed in infill 320 (as seen in FIG. 12) to accommodate structure 312.

An implant bore may be formed across a bone segment before implant 300 is threaded into it. In some embodiments the diameter of the bore is approximately the same as the root diameter of threads 310. In some embodiments, a tap may be used to create internal threads inside the bone before implant 300 is inserted. In other embodiments, self-boring and/or self-tapping features are provided on implant 300. Implant 300 may be threaded into the bore until just the proximal head portion protrudes from the bone. In other implementations, proximal head portion may be partially or fully recessed into the bone, or flush with the outer surface of the bone.

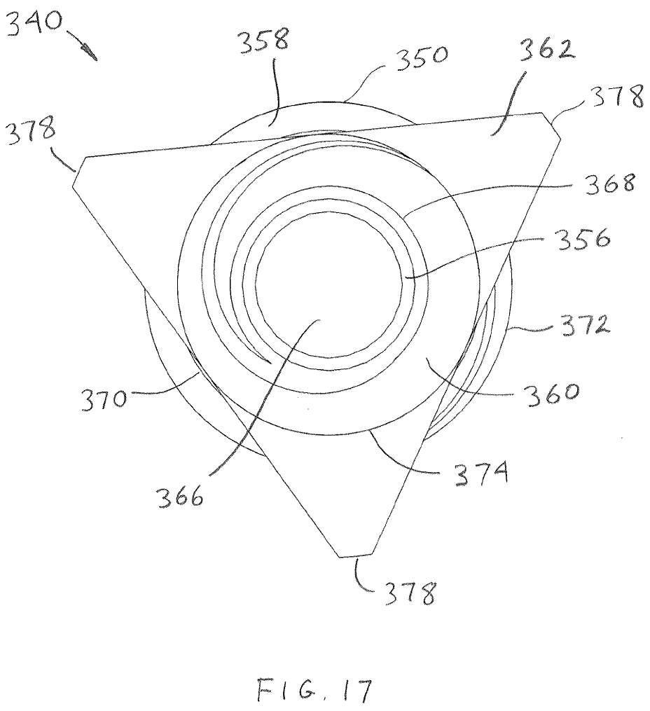

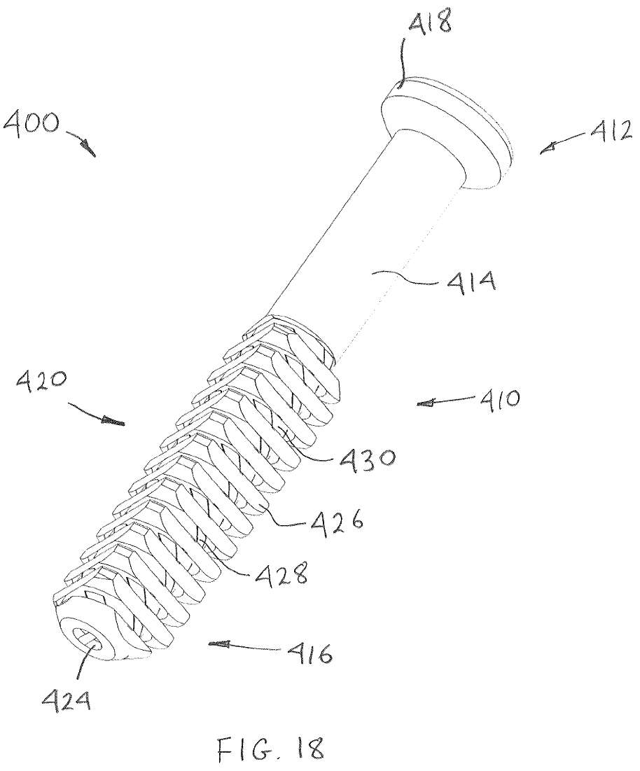

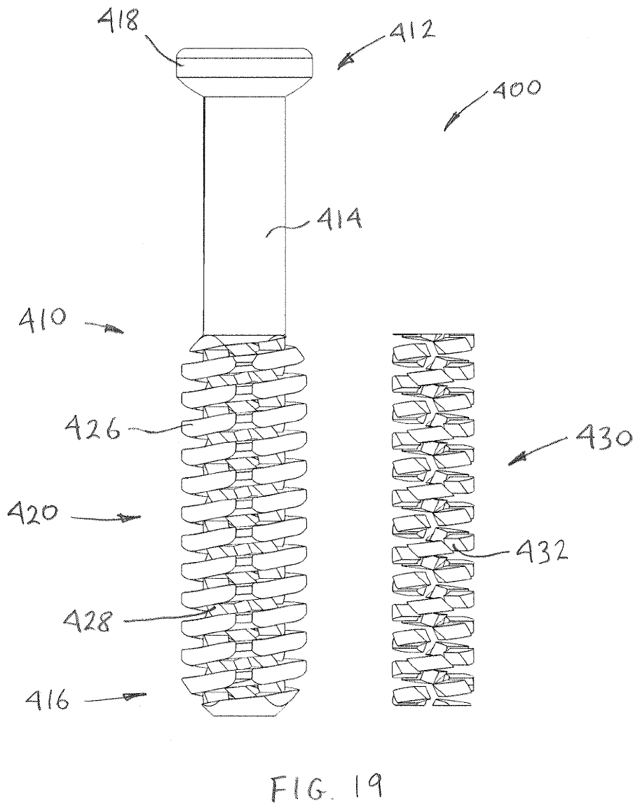

Referring to FIGS. 14-17, another exemplary embodiment of a threaded implant system constructed according to aspects of the present disclosure is shown. Implant 340 comprises a main body 350 having a proximal end 352, a mid-section 354 and a distal end 356. Proximal end 352 is provided with a first set of threads 358 while distal end 356 is provided with a second set of threads 360. As best seen in the exploded view of FIG. 15, mid-section 354 has a constant diameter configured to receive triangle sleeve 362 (as shown in FIG. 14) such that it may rotate with respect to main body 350 while being constrained from axial movement.

Referring to FIG. 16, proximal end 352 of main body 350 may be provided with a hexagonally shaped socket 364 for receiving a drive tool (not shown) when implant 340 is being inserted or removed. A through bore 366 may be provided along the central axis from the proximal end 352 to the distal end 356. As also shown in FIG. 16, main body 350 may be provided with a constant root or minor diameter taper that extends from a smaller diameter 368 at distal end 356 to a larger diameter 370 closer to proximal end 352, except for mid-section 354 which has a smaller diameter than the tapered root or minor diameter sections adjacent to it. The first set of threads 358 may have a constant outer or major diameter 372, such that threads 358 protrude radially less from the root or minor diameter as they extend proximally along main body as shown in FIG. 16. Similarly, the second set of threads 360 may have a constant outer or major diameter 374 (larger than the outer or major diameter 372 of the first set of threads 358), such that threads 360 protrude radially less from the root or minor diameter as they extend proximally along main body as shown in FIG. 16.

In some variations of this embodiment (not shown), the pitch of distal threads 360 is greater than the pitch of proximal threads 372. This arrangement causes implant 340 to advance more rapidly in relation to a distal bone segment that it does relative to a more proximal bone segment, thereby drawing the two bone segments closer together (e.g. compressing a joint between the two bone segments) when the implant is tightened into place.

Referring to FIGS. 14 and 15, one or more fenestrations 376 may be provided in triangle sleeve 362 such that they communicate with a space around mid-section 354. Fenestrations 376 provide additional area for on-growth, ingrowth and through-growth of new bone tissue after implantation of device 340. During implantation, bone chips may be placed inside fenestrations 376 to aid in new bone growth. Chamfers 378 (also shown in FIG. 17) may be provided along the apexes of triangle sleeve 362. One advantage to providing chamfers 378 is that stress concentrations are avoided when creating a bore through the bone for implant 340 to reside in.

Device 340 may be implanted in bone in a manner similar to previously described embodiments. Triangle sleeve 362 may be provided with tapered leading edges (as shown in FIG. 14), self-broaching features (not shown), and/or a separate broaching instrument (not shown) may be used to create a triangularly shaped bore before device 340 is implanted. In some embodiments, triangle sleeve 362 resides across a gap between two bone segments once device 340 is in place. A triangularly shaped bore may be broached just deep enough to accommodate triangle sleeve 362, but not all the way to the distal tip of device 340. In some embodiments, the full depth of threads 358 extends beyond the flats of triangle sleeve 362 and into the surrounding bone, as depicted in FIG. 17. During implantation, triangle sleeve 362 may freely rotate with respect to main body 350 such that it travels axially into the triangularly shaped bore in the bone without rotating while main body 350 is rotated and advances with threads 360 and 358. In some embodiments, bone in-growth and through-growth after implantation causes triangle sleeve 362 to become rigid with main body 350. This mechanism inhibits implant 340 from backing out of the bone or migrating further into it, since triangle sleeve 362 is prevented from rotating by the surrounding triangularly shaped bore in the bone. In some embodiments (not shown), a mechanism such as a toggle, cam, or other feature can be activated immediately after implantation to lock triangle sleeve 362 to main body 350, thereby providing immediate anti-rotation.

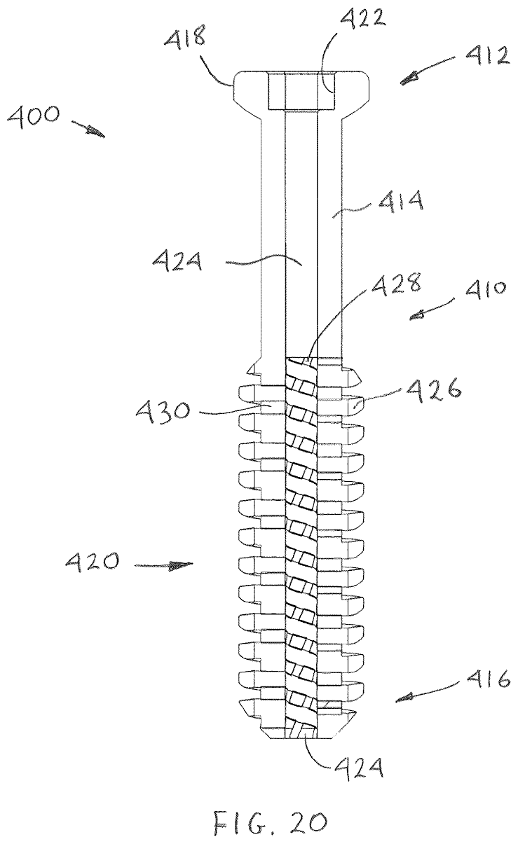

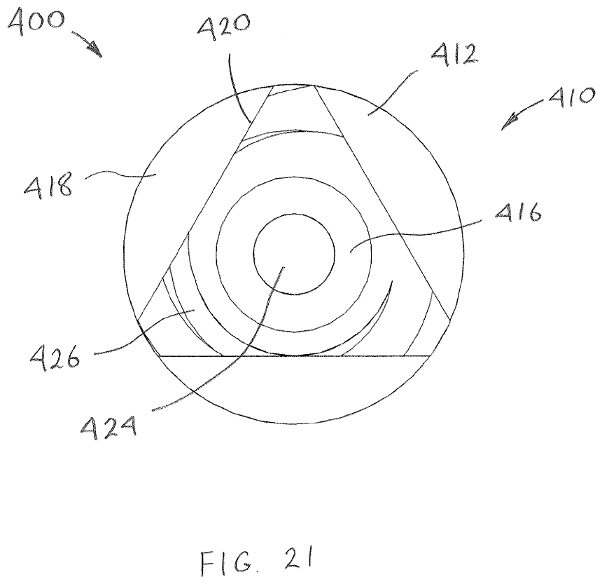

Referring to FIGS. 18-21, another exemplary embodiment of a threaded implant system constructed according to aspects of the present disclosure is shown. Implant 400 comprises a main body 410 having a proximal end 412, a mid-section 414 and a distal end 416. Proximal end 412 is provided with a head portion 418 configured to abut against an outer surface of a bone segment when implant 400 is implanted therein. Distal end 416 is provided with a threaded portion 420 having a triangular cross-section, as best seen in FIGS. 18 and 21. Mid-section 414 may have a constant diameter that extends radially to the same extent as the flat portions of the triangular cross-section. As shown in FIG. 20, proximal end 412 may be provided with a hexagonally shaped socket 422 for receiving a drive tool (not shown) when implant 400 is being inserted or removed. A through bore 424 may be provided along the central axis from the proximal end 412 to the distal end 416.

Similar to previously described implant 300 of FIGS. 11-13, threaded portion 420 of implant 400 is provided with external threads 426 and an internal counter-rotating support structure 428. External threads 426 are used to engage bone when threading implant 400 across a bone joint. In some embodiments, external threads 426 are self-tapping. In this embodiment, internal counter-rotating support structure 428 extends helically in an opposite direction from external threads 426 as shown. Support structure 428 provides stiffness and torsional rigidity to implant 400 while permitting fenestrations between external threads 426 for promoting better bony on-growth, ingrowth and/or through-growth. In this exemplary embodiment, a single external thread 426 helically extends from near the distal end 416 to the mid-section 414 of implant 400, although the thread is interrupted and has portions removed by the flat sides of the triangular cross-section. In other embodiments (not shown), multiple starts of external threads 426 may be employed. In this embodiment, internal counter-rotating support structure 428 comprises four starts. In other embodiments (not shown), fewer or more starts may be employed. In this embodiment, internal counter-rotating support structure 428 has a pitch that is eight times the pitch of external threads 426. In other embodiments (not shown), the pitch of structure 428 may be less or more than eight times that of threads 426, and or may form a shape other than helical.

Also similar to previously described implant 300 of FIGS. 11-13, the fenestrations or interstices of implant 400 between external threads 426 and internal counter-rotating support structure 428 are filled with porous infill 430. Infill 430 provides scaffolding with a large surface area for new bone growth. Infill 430 can also add additional strength to implant 400 in compression, tension, torsion, bending, shear, etc., but still allow for better bony on-growth, ingrowth and/or through-growth than if the fenestrations or interstices were completely filled with less porous material. The exploded view of FIG. 19 shows threaded portion 420 of implant 400 separately from the porous infill 430 for clarity, although in this embodiment infill 430 is a collection of many individual segments rather than an interconnected structure. In some embodiments, threaded portion 420 may also be porous, but having a different porosity than infill 430, as previously described. Both may be made together in the same manufacturing process, such as 3D printing or other additive manufacturing process.

In some embodiments, porous infill 430 has the same inner diameter as that of through bore 424, forming a continuous inner surface as shown in FIG. 20. The outer diameter of infill 430 may be the same diameter of the radially inward end of the tapered portions of threads 426, thereby forming the root diameter of threads 426. In this embodiment, these diameters are also equal to the outer diameter of mid-section 414. The inner and outer diameters of counter-rotating support structure 428 may also be the same as those of infill 430, such that breaks 432 are formed in infill 430 (as seen in FIG. 19) to accommodate structure 428.

In some embodiments, the implant site may be prepared by forming a round bore into the bone having a diameter approximately equal to the root diameter of threads 426. Implant 400 may be provided with self-boring and/or self-tapping features. Implant 400 may be implanted by inserting a hexagonal driver (not shown) into socket 422 and rotating implant 400 until head portion 418 contacts, becomes flush with or recessed within the outer bone surface. Bone chips may be packed into through bore 424 to aid in bone growth to further secure implant 400 as it heals in place.

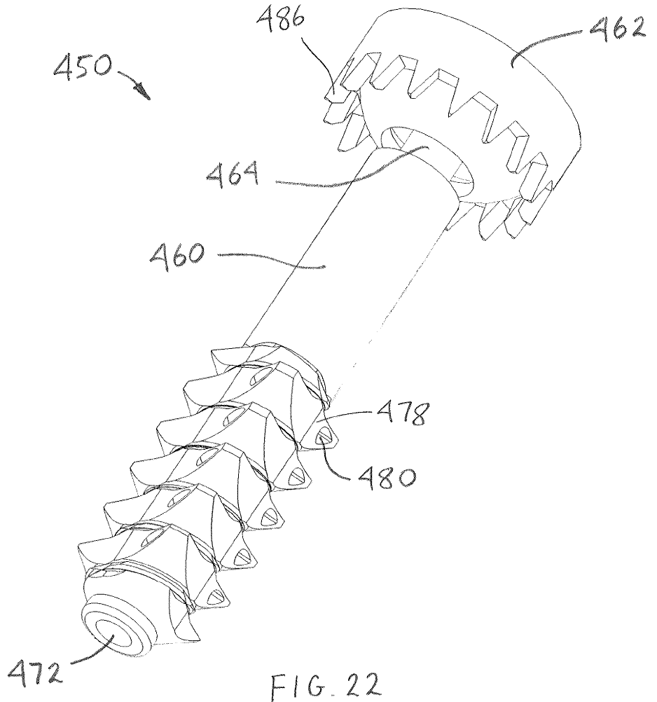

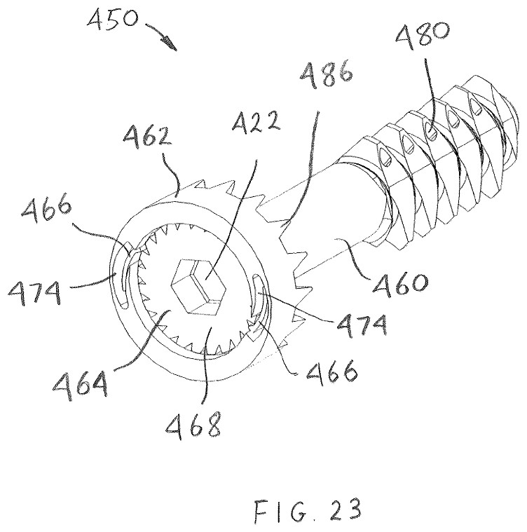

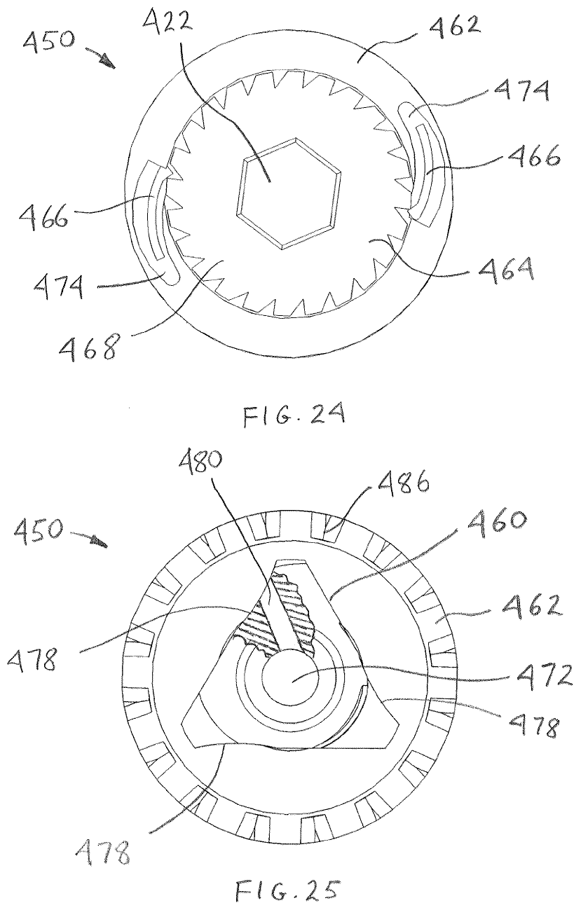

Referring to FIGS. 22-27, another exemplary embodiment of a threaded implant system constructed according to aspects of the present disclosure is shown. As best seen in FIG. 26, implant 450 comprises a main body 460, a proximal screw cap 462, a proximal screw 464, and a pair of rotation stops 466. Proximal screw 464 comprises a head portion 468 and a shaft portion 470. Shaft portion 470 may be provided with external threads (not shown) for threadably engaging with internal threads (not shown) located in the proximal end of central bore 472 which passes through main body 460. When implant 450 is assembled (as best seen in FIGS. 22 and 23), proximal screw cap 462 is captivated between head portion 468 of proximal screw 464 and the proximal end of main body 460. Rotation stops 466 are recessed within curved slots 474 in proximal screw cap 462 (as best seen in FIGS. 23 and 24) such that they prevent or inhibit counter-clockwise rotation of proximal screw head portion 468 relative to proximal screw cap 462.

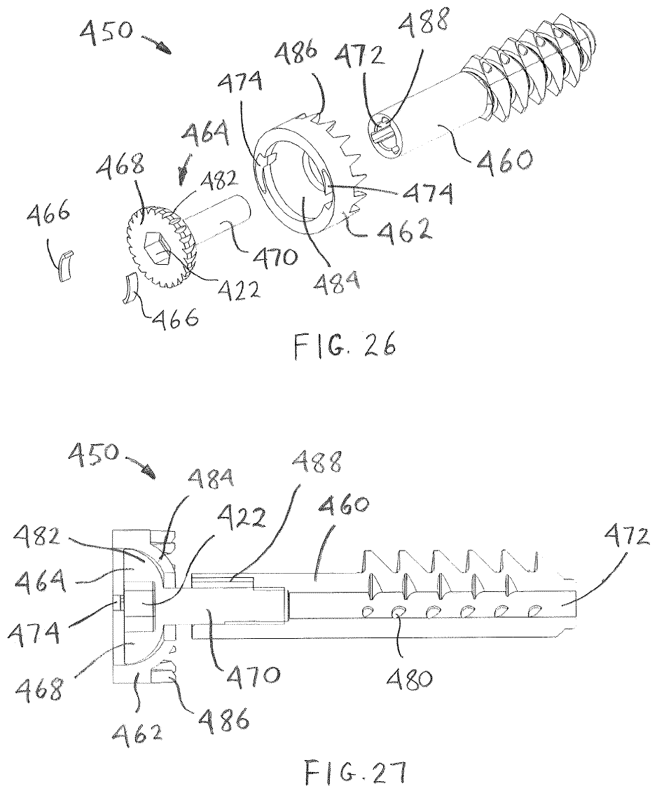

Referring to FIG. 22, the distal end of implant 450 may be provided with threads 476 configured to engage with a bone segment. In this embodiment, as best seen in FIG. 27, the threads 476 have a non-symmetrical longitudinal cross-section that has a saw-tooth pattern. More specifically, the proximal sides of the threads extend in a radial direction (perpendicular to the central axis of implant 450) while the distal sides of the threads are angled relative to the central axis. One advantage to this configuration is that it provides a higher pullout force when implanted in bone. It can also be seen in FIG. 27 that threads 476 get progressively wider as they extend proximally (i.e. the thread peaks get wider and the roots or valleys get narrower.) This arrangement allows for the more proximal threads to press against the adjacent bone grooves more than they otherwise would. The entire threaded area is constantly cutting into un-cut bone and allows the threads to create continuous compression into the bone during advancement.

As best seen in FIG. 25, the threads 476 have a transverse cross-section that is generally triangular in shape. A scoop, divot or hook shape 478 may be provided on the leading edge (when rotating in an insertion direction) of each apex of the triangular-shaped threads. A lateral bore 480 may also be provided to connect each scoop 478 or leading edge with central bore 472. The lateral bores 480, in conjunction with the scoops 478 if provided, serve to harvest bone chips or fragments (not shown) as implant 450 is threaded into place. Bone chips may pass through lateral bores 480 and into central bore 472 where they may accumulate. The bone chips may then be removed through the proximal end of central bore 472 and manually reintroduced around the implant, may be forced out the distal end of the implant with a tamper, pushed back out of lateral bores 480 once implant 450 is in the desired position, and/or may be left in place to promote bony ingrowth into implant 450.

Referring to FIGS. 26 and 27, the proximal side of head portion 468 of proximal screw 464 may be provided with a hexagonally shaped socket 422 for receiving a drive tool (not shown) when implant 450 is being inserted or removed. The distal side of head portion 468 may be provided with a curved, convex portion 482 configured to mate with a curved, concave portion 484 located on proximal screw cap 462. The circumference of the distal side of proximal screw cap 462 may be provided with a series of teeth 486 configured to bite into bone. With this arrangement, proximal screw cap 462 may freely pivot in two dimensions relative to proximal screw 464 so that most or all of the teeth 486 of screw cap 462 can engage with an outer surface of a bone segment when implant 450 is implanted therein, particularly when implant 450 is implanted in an orientation that is not orthogonal to the outer surface of the bone.

In this exemplary embodiment, main body 460 is implanted first without proximal screw cap 462 or proximal screw cap 464. The implant site may be prepared in a manner similar to those of previously described embodiments. A three-lobed driver tool (not shown) may be inserted into a mating three-lobe receptacle 488 (shown in FIG. 26) located at the proximal end of main body 460. Main body 460 may then be threaded into the bone to a desired height, for example, such that the proximal end of main body 460 is just below the outer surface of the bone. The driver tool is then removed from main body 460. Bone chips may then be packed into or moved inside central bore 472. Proximal screw cap 462 may then be placed over the implant bore or over the proximal screw 464, and the distal end of proximal screw 464 may be threaded into the proximal end of main body 460. A hexagonal driver tool (not shown) may be used to tighten proximal screw cap 462 against the outer surface of the bone. Rotation stops 466 inhibit proximal screw 464 from backing out, as previously described. In some embodiments, rotation stops 466 may be removed from proximal screw cap 462 after implantation to allow proximal screw 468 to be rotated in the opposite direction to remove implant 450.

In some embodiments (not shown), a compression spring may be provided between the proximal screw head or proximal screw cap and the proximal bone surface to maintain compression force on the joint and/or to inhibit the implant from backing out of the bone ..

In any of the previously described embodiments, various thread profiles may be utilized. For example, a buttress, V, square, multi start, tapered (root and width), variable pitch, or other thread profile may be used. In some designs, the ISO 5835 standard from the American National Standards Institute (ANSI) may be used for guidance. Thread profiles may be designed to reduce stress concentrations. Variable thread depths may be used.

Materials that may be used to form the implants include: titanium alloy, stainless steel, ceramic, other alloys, polymers, and bone. In some embodiments, the implant or portions of the implant are additively manufactured. Porosity, fenestrations, nano tubes, nano surface treatments, hydroxyapatite, drug elution, bioactive and anti-microbial (e.g. silver) materials, coatings and/or treatments may be used to encourage bone growth and/or deliver therapeutic benefits. Porosity may vary radially, longitudinally, and/or in other manners. The implants may include expandable sections and may include a modular design.

The implants disclosed herein may be provided in a various incremental lengths to match various anatomies. In some implementations, the lengths range from 30 to 160 mm. In some implementations, various incremental diameters may be provided, such as 6 to 18 mm. In some implementations, the thread pitch is 1.5 to 10 mm. In some implementations, the pore size of some or all of the implant is 250 to 1000 microns. In some implementations, the porosity is 50 to 80%.

In some implementations, a threaded proximal portion configured to engage the ilium is 10 to 15 mm long, a triangular middle portion without threads is 10-15 mm long, and a distal threaded portion configured to engage the sacrum is provided in various lengths, depending on the anatomy of the particular implant site.

In any of the previously described embodiments, the external threads may get progressively wider as they extend proximally. This arrangement allows for the more proximal threads to press against the adjacent bone grooves more than they otherwise would to provide a tighter fit of the implant against the bone.

In some embodiments, the implants disclosed herein are specifically designed to accommodate four or five zones of the sacroiliac joint. These zones can include: 1) the lateral iliac wall; 2) the ilium; 3) the SI joint itself; 4) the sacral ala; and 5) sacral vertebral body. For example, the implant design may include 1) a mechanism to lock against the lateral iliac wall (such as a series of teeth 486 on a proximal head as previously described), 2) a finer thread configured to engage the ilium, 3) fenestrations configured to be located inside the SI joint itself when the device is implanted to promote bone ingrowth and/or deliver biologics, 4) a course thread for the ala, and 5) possibly a fine thread for the sacral body. Other suitable features may also be provided and configured for each of the zones, particularly the first four zones.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the disclosure as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. (canceled)

2. An elongated threaded implant sized and configured for implantation in a sacroiliac joint of a patient, comprising:
an internal support extending axially from a proximal end of the threaded implant to a distal end of the implant;
an external thread extending radially outward relative to the internal support and extending axially along a length of the implant; and
a porous infill disposed in a helical configuration about an implant axis between the external thread such that the porous infill forms a root diameter of the external thread,
the porous infill comprising a plurality of pores sized to provide for one or more of bony on-growth, ingrowth or through-growth,
the porous infill comprising a plurality of breaks in the helical configuration,
wherein the plurality of breaks are in a helical configuration that is different than the helical configuration of the porous infill between the external thread,
wherein a path defined by the helical configuration of the plurality of breaks is void of the porous infill,
wherein the path has a pitch that is at least eight times a pitch of the helical configuration of the porous infill between the external thread, and
wherein the external thread and the helically configured porous infill are manufactured from the same material, and are manufactured together during the same additive manufacturing process.

3. The threaded implant of claim 2, further comprising a through bore extending along a central axis of the threaded implant.

4. The threaded implant of claim 2, wherein the helically configured porous infill is at least 20% more porous than the external thread.

5. The threaded implant of claim 2, wherein the external threads comprise a first set of distal threads and a second set of proximal threads located proximally to the first set of threads along the implant axis.

6. The threaded implant of claim 5, wherein the first set of threads has a greater pitch than the second set of threads.

7. The threaded implant of claim 2, wherein the implant is devoid of the porous infill in a region located between the proximal end and the distal end of the implant.

8. The threaded implant of claim 7, wherein the region devoid of porous infill is positioned along the implant axis such that it at least partially resides in between a patient's ilium and sacrum once implanted.

9. The threaded implant of claim 2, wherein the proximal end of the implant is provided with a head portion.

10. The threaded implant of claim 9, wherein a proximal side of the head portion is provided with a recess configured to mate with a driving tool to screw the implant into bone.

11. The threaded implant of claim 9, wherein a distal side of the head portion is provided with a partially spherical surface configured to mate with another component such that the other component may freely pivot in two dimensions relative to head portion.

12. An elongated threaded implant sized and configured for implantation in a sacroiliac joint of a patient, comprising:
an internal support extending axially from a proximal end of the threaded implant to a distal end of the implant;
an external thread extending radially outward relative to the internal support and extending axially along a length of the implant; and
a scaffolding disposed in a helical configuration about an implant axis between the external thread such that the scaffolding forms a root diameter of the external thread,
the scaffolding comprising a plurality of pores sized to provide for one or more of bony on-growth, ingrowth or through-growth,
the scaffolding comprising a plurality of breaks in the helical configuration,
wherein the plurality of breaks are in a helical configuration that is different than the helical configuration of the scaffolding between the external thread,
wherein a path defined by the helical configuration of the plurality of breaks is void of the scaffolding,
wherein the path has a pitch that is at least eight times a pitch of the helical configuration of the scaffolding between the external thread, and
wherein the external thread and the helically configured scaffolding are manufactured from the same material, and are manufactured together during the same additive manufacturing process.

13. The threaded implant of claim 12, further comprising a through bore extending along a central axis of the threaded implant.

14. The threaded implant of claim 12, wherein the helically configured scaffolding is at least 20% more porous than the external thread.

15. The threaded implant of claim 12, wherein the external threads comprise a first set of distal threads and a second set of proximal threads located proximally to the first set of threads along the implant axis.

16. The threaded implant of claim 15, wherein the first set of threads has a greater pitch than the second set of threads.

17. The threaded implant of claim 12, wherein the implant is devoid of the scaffolding in a region located between the proximal end and the distal end of the implant.

18. The threaded implant of claim 17, wherein the region devoid of scaffolding is positioned along the implant axis such that it at least partially resides in between a patient's ilium and sacrum once implanted.

19. The threaded implant of claim 12, wherein the proximal end of the implant is provided with a head portion.

20. The threaded implant of claim 19, wherein a proximal side of the head portion is provided with a recess configured to mate with a driving tool to screw the implant into bone.

21. The threaded implant of claim 19, wherein a distal side of the head portion is provided with a partially spherical surface configured to mate with another component such that the other component may freely pivot in two dimensions relative to head portion.

22. A method of implanting an elongated threaded implant in a sacroiliac (SI) Joint of a patient, the method comprising:
providing an elongated threaded implant comprising:
an internal support extending axially from a proximal end of the threaded implant to a distal end of the implant;
an external thread extending radially outward relative to the internal support and extending axially along a length of the implant; and
a porous infill disposed in a helical configuration about an implant axis between the external thread such that the porous infill forms a root diameter of the external thread,
the porous infill comprising a plurality of pores sized to provide for one or more of bony on-growth, ingrowth or through-growth,
the porous infill comprising a plurality of breaks in the helical configuration,
wherein the plurality of breaks are in a helical configuration that is different than the helical configuration of the porous infill between the external thread,
wherein a path defined by the helical configuration of the plurality of breaks is void of the porous infill,
wherein the path has a pitch that is at least eight times a pitch of the helical configuration of the porous infill between the external thread, and
wherein the external thread and the helically configured porous infill are manufactured from the same material, and are manufactured together during the same additive manufacturing process, and
using a driving tool to screw the implant across the SI-Joint using a postero-lateral approach, entering from a posterior iliac spine of an ilium of the patient, angling through the SI-Joint, and terminating in the patient's sacral alae.

23. The method of claim 22, wherein the step of screwing the implant across the SI-Joint comprises crossing at least three cortical walls.
